(19) United States
(12) Patent Application Publication
(10) Pub. No.: US 2009/0318715 A1
(43) Pub. Date: Dec. 24, 2009

(54) Method for the Production of D,L-2-Hydroxy-4-Alkylthio Butyric Acid

(76) Inventors: Patrick Deck, Mannheim (DE); Kai Michael Exner, Oberursel (DE); Boris Buschhaus, Mannheim (DE)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/438,192

(22) PCT Filed: Aug. 15, 2007
(86) PCT No.: PCT/EP07/58426

§ 371 Date: Jun. 16, 2009

(30) Foreign Application Priority Data
Aug. 24, 2006 (EP) 06119485.8

(51) Int. Cl. C07C 319/02; C07D 307/33

(52) U.S. Cl. 549/313; 562/581

(57) ABSTRACT

The present invention relates to a process for preparing compounds of the formula (I)

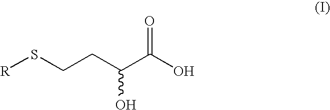

by reacting compounds of the formula (II)

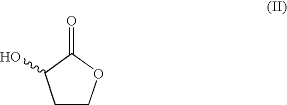

with thiolates (RS)ₙM.

The present invention further relates to a process for preparing compounds of the formula (II) from γ-butyrolactone.

The present invention relates to a process for preparing compounds of the formula (I)

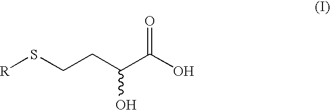

where R is C₁- to C₆-alkyl.

The present invention further relates to a process for preparing compounds of the formula (II)

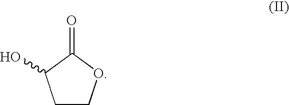

Methionine and methionine hydroxy analog are, besides L-glutamic acid and L-lysine, among the economically most important amino acids. The economic importance of methionine derives from the feedstuff-saving rearing of productive livestock.

Methionine is an essential sulfur-containing amino acid whose metabolically active form is S-adenosylmethionine (SAM).

Methionine (D,L-2-amino-4-methylthiobutyric acid) can, in contrast to all other amino acids, be utilized fully even as racemate by the organism. The body is able to convert the D form completely into the active L form. Thus, in an industrial synthesis the configuration of the α-amino group is immaterial.

It is of interest that the organism is also able to utilize methioninehydroxy analog (D,L-2-hydroxy-4-methylthiobutyric acid, MHA) as complete substitute for methionine. The amino group of methionine is replaced in MHA by a hydroxyl group. In this case too, conversion into the active L form of methionine takes place in the body. Thus, industrially manufactured racemic MHA also represents a complete substitute for methionine.

The processes for preparing methionine and MHA in feedstuff quality are based substantially on acrolein, methyl mercaptan and hydrocyanic acid as precursors.

A process described in DE 1 906 405 starts in a first stage from acrolein and mercaptan, which are reacted to give 3-methylmercaptopropionaldehyde (MMP). This is reacted in a next step with hydrocyanic acid and ammonium bicarbonate to give a hydantoin which is subsequently converted by alkali into potassium D,L-methionate. Acidification affords D,L-methionine.

Likewise starting from MMP, according to U.S. Pat. No. 2,745,745 reaction with hydrocyanic acid in the presence of sodium hydroxide at 35-40° C. results in a cyanohydrin. Hydrolysis by strong mineral acids such as sulfuric acid affords the amide as intermediate, and finally MHA. Ammonium bisulfate is formed as byproduct.

DE 840 996 discloses a process for producing thioethercarboxylic acids. This entails unsubstituted lactones or lactones having aromatic radicals, such as phthalides or coumarins, being heated with alkali metal or alkaline earth metal compounds of mercapto compounds which comprise no unesterified carboxyl groups. The reaction takes place without addition of solvent, if appropriate with an excess of lactone as solvent or in the presence of inert solvents such as benzene, toluene or decalin.

The object was, starting from starting materials of lower toxicity, to find a cost-effective process for preparing D,L-2-hydroxy-4-alkylthiobutyric acid of the formula (I)

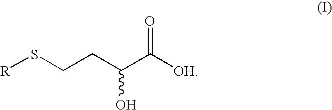

In particular, the object was, starting from starting materials of lower toxicity, to find a cost-effective process for preparing MHA of the formula (Ia)

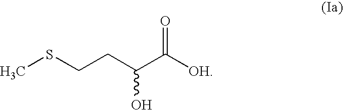

The object has been achieved according to the invention by provision of a process for preparing compounds of the formula (I)

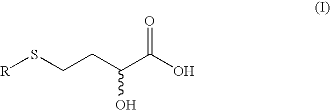

which comprises reacting compounds of the formula (II)

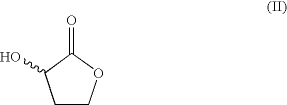

with thiolates RSM.

R means in this connection according to the invention C₁- to C₆-alkyl.

Examples thereof are methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, 1,1-dimethylpropyl, 1,2-dimethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1-ethyl-1-methylpropyl, 1-ethyl-3-methylpropyl and mixtures thereof.

The radicals may also comprise one or more stereocenters.

R is preferably C₁- to C₄-alkyl.

Examples thereof are methyl, ethyl, n-propyl, 1-methylethyl, n-butyl, 1-methylpropyl, 2-methylpropyl, 1,1-dimethylethyl and mixtures thereof.

The radicals may comprise at least one stereo center.

R is particularly preferably methyl. In this case, the compound of the formula (I) is MHA of the formula (Ia)

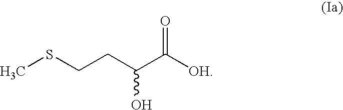

M in the thiolates (RS)ₙM is alkali metal, alkaline earth metal, Fe, Zn or a mixture thereof.

Alkali metal is Li, Na, K, Rb, Cs or a mixture thereof.

Alkaline earth metal is Be, Mg, Ca, Sr, Ba or a mixture thereof.

Where M is alkali metal, n is equal to 1.

Where M is alkaline earth metal, Zn or a mixture thereof, n is equal to 2.

Where M is Fe, n is equal to 2 and/or 3.

M is preferably Li, Na, K or a mixture thereof, and n is preferably equal to 1.

For a given M which may be alkaline earth metal, Zn or Fe, the radicals R in the corresponding thiolate (RS)ₙM may be identical or different.

Thiolates of the formula (RS)ₙM with identical or different radicals R and/or identical or different metals M can be employed simultaneously.

Preferably only one thiolate of the formula (RS)ₙM is employed.

In all the formulae hereinbefore and hereinafter, the wavy line represents an S or R configuration at the relevant carbon atom. A formula comprising a wavy line preferably represents any mixture, particularly preferably a racemic mixture, of the enantiomeric forms of the compound. Alternatively, such a formula may represent a particular enantiomeric form which is not precisely specified.

A carbon atom having four different substituents is a stereo center. If a molecule has exactly one stereo center, two different configurations of the corresponding molecule are possible. The two non-superimposable mirror-image forms of such a molecule are referred to as enantiomers. R and S enantiomers are distinguished according to the rules of Cahn, Ingold and Prelog.

A mixture with equal proportions of the two enantiomers is called racemate or racemic mixture. The molar ratio and the ratio by weight of the two enantiomers in the racemate are identical because the enantiomers have the same molecular mass.

In the context of this application, reference is made only to the configuration at the carbon atom α to the acid or ester group in order to determine whether a mixture of isomers, a racemate as specific case, or one enantiomer is present. If other stereo centers are present on the radical R, they have no relevance to these statements about the stereochemistry.

The thiolates (RS)ₙM can be employed as solutions. In this connection, the concentration of the thiolates (RS)ₙM is typically 10% by weight or more, preferably 20% by weight or more. It is also possible to employ solutions with a concentration of 50% by weight or more, preferably 90% by weight or more. It is moreover possible to employ the thiolates (RS)ₙM in particular as solution in the corresponding thiol (RS)ₙH.

One advantage of the invention is that the stereoisomerism of the hydroxy group a to the cyclic ester group in the compounds of the formula (II) is retained in the preparation of the compounds of the formula (I). Normally, racemic mixtures are employed as compounds of the formula (II), so that the correspondingly obtained compounds of the formula (I) are also racemic mixtures.

If, however, one stereoisomeric form of the compound of the formula (II) predominates, then the compound of the formula (I) obtained therefrom is likewise predominantly in this stereoisomeric form.

If a racemic mixture is not employed, a further preferred embodiment of the present invention is for one of the stereoisomeric forms to clearly predominate.

In the case of a mixture of isomers, the enantiomeric excess of the mixture of isomers employed is preferably at least 90%.

The enantiomeric excess is defined as

$$\% ee = \vert \frac{[ R ] - [ S ]}{[ R ] + [ S ]} \vert ,$$

where
[R]: Concentration of the R isomer;
[S]: Concentration of the S isomer.

In a further preferred embodiment, the compound of the formula (II) is employed in enantiopure form.

Where mixtures of isomers of compounds of the formula (II) in which one of the enantiomeric forms predominates are employed, the process of the invention results in compounds of the formula (I) in which one of the enantiomeric forms likewise predominates.

If one of the enantiomeric forms of the compound of the formula (II) is present exclusively, i.e. the corresponding compound is enantiopure, the process of the invention results in a compound of the formula (I) which is likewise enantiopure.

The process of the invention preferably takes place in polar aprotic solvents.

The polarity of a solvent is quantified via its molecular dipole moment which is connected to the macroscopic permittivity. Thus, when the value of the permittivity of a solvent is known it is possible to make statements about its polarity. Values of permittivity can be found for example in the Handbook of Chemistry and Physics, 76^{th} edition, 1995, CRC Press, Inc., Boca Raton.

A polar solvent generally has a value of the permittivity of 10 or more, preferably 20 or more, particularly preferably 40 or more, at a temperature of 293.2 K.

A solvent is referred to as aprotic if it is unable or is able only with difficulty to eliminate protons because either it comprises no hydrogen atoms or the hydrogen bonds have a high covalent character. One measure of the ability of protons to be eliminated from compounds is the acid strength Kₐ. This is determined in water, unless indicated otherwise. Normally, the negative decadic logarithm of the acid strength, the pKₐ, is indicated.

An aprotic solvent generally has a pKₐ or, in the case of a plurality of protons which can possibly be eliminated, a lowest pKₐ of 20 or more, preferably of 22 or more, particularly preferably of 24 or more, at a temperature of 293.2 K.

Solvents can be employed pure or as mixture.

Polar aprotic solvents can be employed as mixture with other solvents, e.g. polar protic solvents or apolar solvents. In this case, the proportion of one or other of the solvents in the solvent mixture usually does not exceed 10% by weight.

Solvents to be preferably employed according to the invention are for example dimethyl sulfoxide, N-methylpyrrolidone or mixtures thereof.

The process of the invention takes place at temperatures which ensure that the reaction proceeds sufficiently quickly. The reaction expediently takes place at temperatures from 50° C. to 200° C.

The compounds of the formula (II) employed in the process of the invention are known to the skilled worker. Concerning these, see Beilsteins Handbuch der Organischen Chemie, Springer Verlag, Ergänzungswerk I, Volume XVIII, p. 296; Ergänzungswerk II, Volume 18, p. 3; Ergänzungswerk III, Volume 18, p. 3; Ergänzungswerk III/IV, Volume 18, p. 3; Ergänzungswerk V, Volume 18, p. 3 and the literature indicated therein.

For the process of the invention, these are preferably obtained from γ-butyrolactone (formula II).

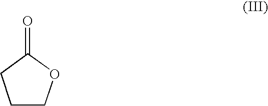

γ-Butyrolactone is available in large quantities as part of the value chain of so-called Reppe chemistry. γ-Butyrolactone is obtained starting from acetylene and formaldehyde via the intermediates 1,4-butyndiol, 1,4-butenediol and 1,4-butanediol.

In a further embodiment, the process of the invention for preparing compounds of the formula (I) includes a preceding process step in which γ-butyrolactone are converted into compounds of the formula (II).

For this purpose, preferably γ-butyrolactone is converted in a first step into compounds of the formula (IV).

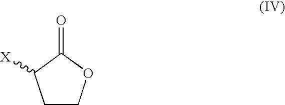

The invention thus further relates to a process in which γ-butyrolactone of the formula (III) is initially converted into compounds of the formula (IV), and the compounds of the formula (IV) are converted in a subsequent step into compounds of the formula (II). The radical X is in this connection according to the invention a halogen atom. It is possible according to the invention for a compound of the formula (IV) always to comprise the same radical X or different X radicals. Halogen means according to the invention fluorine, chlorine, bromine and/or iodine. Chlorine or bromine are preferred. Chlorine is particularly preferred.

In a preferred embodiment of the process of the invention for preparing compounds of the formula (I) by reacting compounds of the formula (II) with thiolates (RS)ₙM, the compounds of the formula (II) are obtained by initially converting γ-butyrolactone of the formula (III) into compounds of the formula (IV), and converting the compounds of the formula (IV) in a subsequent step into compounds of the formula (II).

α-Bromo-γ-butyrolactone can be obtained by reacting bromine Br₂ with γ-butyrolactone at about 100° C. in the presence of phosphorus tribromide PBr₃. The resulting bromo compound is isolated if appropriate, but is preferably not isolated and is immediately reacted further with barium hydroxide to give α-hydroxy-γ-butyrolactone. Barium hydroxide is normally employed as Ba(OH)₂.8H₂O.

Phosphorus tribromide is preferably employed in amounts of from 1 to 20 mol %, further preferably from 5 to 15 mol %, based on γ-butyrolactone. In a particularly preferred embodiment, phosphorus tribromide is employed in an amount of 10 mol % based on γ-butyrolactone.

Phosphorus tribromide is ordinarily added to the γ-butyrolactone at temperatures from −10 to +10° C. A suitable solvent is present if appropriate, but preferably no solvent is present. Bromine is generally likewise added at a temperature from −10 to +10° C. Bromine is usually employed in amounts of from 100 to 150 mol %, preferably from 110 to 140 mol %, based on γ-butyrolactone. In a particularly preferred embodiment, bromine is employed in an amount of 130 mol % based on γ-butyrolactone.

After addition of the bromine, the reaction mixture is ordinarily heated for a certain period, e.g. for one to ten hours. The temperatures in this case are ordinarily in the range from 80 to 150° C.

Excess bromine is preferably reduced after the reaction. This takes place for example by adding NaHSO₃ solution.

α-Chloro-γ-butyrolactone can be obtained by chlorinating γ-butyrolactone without adding a catalyst at elevated temperatures which are for example 100-200° C., preferably 140-160° C. Byproducts which may be formed in this case are α,α-dichloro-γ-butyrolactone and 2,4-dichlorobutyric acid.

The 2,4-dichlorobutyric acid is preferably not removed for further reaction, because the cyclic form of α-hydroxy-γ-butyrolactone is formed again in the alkaline hydrolysis. The α,α-dichloro-γ-butyrolactone can preferably be removed by distillation.

Hot barium hydroxide solution can be used to convert α-chloro-γ-butyrolactone and 2,4-dichlorobutyric acid into α-hydroxy-γ-butyrolactone.

Chlorine is usually employed in amounts of from 100 to 150 mol %, preferably from 110 to 140 mol %, based on γ-butyrolactone. In a particularly preferred embodiment, chlorine is employed in an amount of 130 mol % based on γ-butyrolactone.

Purification by nitrogen flushing and/or washing with water is possible.

The distillation preferably takes place under a reduced pressure, for example an absolute pressure of 1 mbar or less, preferably under 10⁻¹ mbar or less, particularly preferably under 10⁻² mbar or less. The product is distilled more than once if appropriate.

The reaction conditions for treating α-chloro-γ-butyrolactone with barium hydroxide are analogous to those for treating α-bromo-γ-butyrolactone with barium hydroxide.

All the processes of the invention can be carried out on various scales batchwise, semicontinuously or continuously. For example, the product can be produced in discontinuous processes in amounts of from 1 g to 1000 tons per batch, preferably 100 kg to 10 tons, and in the case of continuous processes with throughputs of from 1 g to 1000 tons per hour, preferably from 100 kg to 10 tons per hour. Specific embodiments are the laboratory scale, the pilot-plant scale, the pilot-plant scale and the production scale. In batchwise processes, the starting materials are fed under the stated conditions into a suitable container and reacted there. The resulting product remains in the reactor. It can be further purified there if appropriate. Alternatively, it can be transferred into other suitable containers such as, for example, distillation columns and further purified there. In continuous processes, the starting materials are fed under the stated conditions into a suitable container and reacted there. The resulting product is removed from the reactor during this and further purified if appropriate.

Semicontinuous processes comprise continuous and batchwise process steps.

Suitable containers for the processes may be for example containers made of glass, steel or stainless steel, which are coated if appropriate. The containers are normally equipped with an appropriate possibility for stirring, such as, for example, magnetic stirrer or anchor stirrer. If desired, the containers can be heated in a suitable manner for example by oil baths or heating jackets operated electrically or by steam. The containers are chosen so that they withstand the temperature and pressure conditions prevailing during the reaction.

Purification can take place in a known manner, for example by distillation. If appropriate, unreacted starting material is returned to the process at a suitable point.

The present invention offers a simple way of obtaining D,L-2-hydroxy-4-alkylthiobutyric acids such as MHA, one of the most economically important amino acids. It is moreover possible to employ γ-butyrolactone as low-cost, easily available and non-toxic starting material which is converted into the desired final product in a few process steps.

The process of the invention is explained in more detail in the following examples. The examples in this case implement the claims and the description further without restricting them in any way.

A) Synthesis of D,L-2-hydroxy-4-methylthiobutyric Acid (MHA)

α-Hydroxy-γ-butyrolactone and sodium methylthiolate NaSCH₃ were introduced into 20 ml of solvent (see table 1) and heated at the reaction temperature indicated in table 1 for a plurality of hours (reaction time). After cooling, the solvent was removed and the residue was taken up in 1N HCl. The solution was extracted with methyl tert-butyl ether, and the combined organic phases were dried over MgSO₄ and evaporated to dryness.

Amounts employed, reaction times, solvents and yields are to be found in table 1.

The yield was determined by final weighing. The purity of the product was analyzed by ¹H-NMR.

TABLE 1

| | | Sodium | | | | |
|---|---|---|---|---|---|---|
| | α-Hydroxy-γ- | methyl- | | Reaction | Reaction | |
| | butyrolactone | thiolate | | time | temperature | MHA yield |
| Experiment | [g (mmol)] | [g (mmol)] | Solvent | [h] | [° C.] | [%] |
| 1 | 1.5 (14.7) | 0.7 (14.7) | DMSO | 16 | 120 | 78 |
| 2 | 1.5 (14.7) | 0.7 (14.7) | DMSO | 30 | 120 | 96 |
| 3 | 1.0 (9.8) | 0.5 (10) | Methanol | 10 | 65 | 11 |
| 4 | 1.5 (14.7) | 0.7 (14.7) | Methanol | 20 | 65 | 30 |
| 5 | 1.0 (9.8) | 0.5 (10) | Acetonitrile/ | 20 | 65 | 34 |
| | | | methanol | | | |
| | | | (1/5)¹⁾ | | | |
| 6 | 1.0 (9.8) | 0.5 (10) | DMSO | 3 | 189 | 100 |
| 7 | 1.0 (9.8) | 0.5 (10) | DMF | 3 | 153 | 100 |

¹⁾Ratio by volume
DMSO: Dimethyl sulfoxide
DMF: Dimethylformamide

B) Synthesis of α-hydroxy-γ-butyrolactone

### Experiment 8 (According to the Invention):

9.5 g (0.035 mol) of PBr₃ were slowly added to 30 g (0.348 mol) of γ-butyrolactone at 0° C. Then, over a period of 3 h, 71.9 g (0.450 mol) of Br₂ were slowly added dropwise. After the solution had been heated at 99° C. for 6 h, H₂O was added and the bromine residues were reduced with a little NaHSO₃ solution. Thereafter 220 g (0.7 mol) of Ba(OH)₂.8H₂O were added and the solution was heated at 100° C. for 15 h. The barium was precipitated with conc. H₂SO₄, the precipitate was filtered off with suction, and the solution was evaporated to dryness. The solid was taken up in ethanol and insolubles were removed. The EtOH was removed and the remaining solid was distilled at 110° C. (6·10⁻³ mbar), with marked elimination of water occurring.

The resulting colorless oil was distilled once again to result in 8.2 g (0.08 mol, yield: 23%) of D,L-α-hydroxy-γ-butyrolactone.

### Experiment 9 (According to the Invention):

28 g (0.32 mol) of γ-butyrolactone were introduced into the apparatus and then a slow stream of chlorine gas was passed through at 125-140° C. After 23 g (0.32 mol) of chlorine had been added, the apparatus was flushed with nitrogen in order to drive out the remaining chlorine gas. After cooling, the crude product was washed with H₂O and distilled.

The resulting α-chlorobutyrolactone was converted into MHA by the method described in the example for the preparation of α-bromobutyrolactone.

The resulting colorless oil was redistilled to result in 7.1 g (0.07 mol) of D,L-α-hydroxy-γ-butyrolactone (yield: 22%).

1. A process for preparing at least one compound of the formula (I) 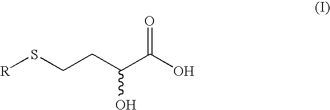
where R is C₁- to C₆-alkyl,
which comprises reacting compounds of the formula (II) 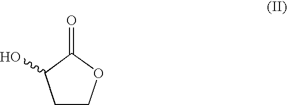
with at least one thiolate (RS)ₙM,
where R has the meaning as in formula (I), and
M is alkali metal, alkaline earth metal, Fe and/or Zn, and
n is 1 if M is alkali metal,
n is 2 if M is alkaline earth metal and/or Zn,
n is 2 and/or 3 if M is Fe.

2. The process according to claim 1, where R is C₁- to C₄-alkyl.

3. The process according to claim 2, where R is methyl.

4. The process according to claim 1, where M is Li, Na and/or K.

5. The process according to claim 1, where M is Na.

6. The process according to claim 1, where the compounds of the formula (II) are employed as enantiomeric mixtures or enantiopure.

7. The process according to claim 1, where the compounds of the formula (II) are employed as racemic mixtures.

8. The process according to claim 1, where the reaction takes place in polar aprotic solvents.

9. The process according to claim 8, where dimethyl sulfoxide, N-methylpyrrolidone or mixtures thereof are employed as solvents.

10. The process according to claim 1 including a preceding process step in which γ-butyrolactone is converted into compounds of the formula (II).

11. A process for preparing compounds of the formula (II), which comprises initially converting γ-butyrolactone into compounds of the formula (IV) 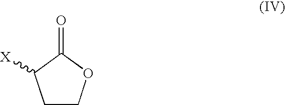
where X is halogen,
and converting the compounds of the formula (IV) in a subsequent substep into compounds of the formula (II).

12. The process according to claim 11, wherein γ-butyrolactone is initially converted into compounds of the formula (IV) 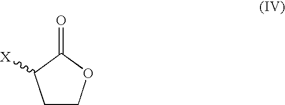
where X is halogen,
and the compounds of the formula (IV) are converted in a subsequent substep into compounds of the formula (II).

14. The process according to claim 12, where X is Cl.

15. A process for preparing at least one compound of the formula (I) 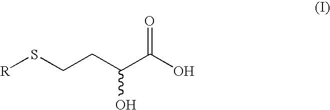
where R is C₁- to C₆-alkyl,
which comprises reacting compounds of the formula (II) 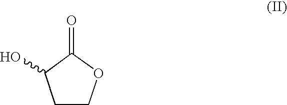
with at least one thiolate (RS)ₙM,
where R has the meaning as in formula (I), and
M is alkali metal, alkaline earth metal, Fe and/or Zn, and
n is 1 if M is alkali metal,
n is 2 if M is alkaline earth metal and/or Zn,
n is 2 and/or 3 if M is Fe
in which the conversion of γ-butyrolactone into compounds of the formula (II) takes place by a process according to claim 11.
